(19) United States
(12) Patent Application Publication
(10) Pub. No.: US 2020/0310557 A1
(43) Pub. Date: Oct. 1, 2020

(54) MOMENTUM-BASED IMAGE NAVIGATION

(71) Applicant: GE Precision Healthcare LLC, Wauwatosa, WI (US)

(72) Inventors: Lauren Parkos, Chicago, IL (US); James Gualtieri, Pittsburgh, PA (US)

(21) Appl. No.: 16/368,496

(22) Filed: Mar. 28, 2019

(51) Int. Cl. G06F 3/0354; G06F 3/0481; G16H 30/20; G06F 3/0485; G06F 3/038

(52) U.S. Cl. CPC G06F 3/0354; G06F 3/0481; G06F 3/0236; G06F 3/0485; G06F 3/0383; G16H 30/20

(57) ABSTRACT

Apparatus, systems, and methods to navigate through a set of z-stacked images are disclosed. An example apparatus includes a position tracker to track movement of a pointing device with respect to a set of z-stacked images, a momentum detector to identify momentum applied to the pointing device in a first interaction based on a speed of navigation through the set of z-stacked images and configure navigation through the set of z-stacked images in a momentum-based mode based on the speed of navigation through the set of z-stacked images, and a navigation mode detector to detect a second interaction with the pointing device and exit a navigation mode and enter a normal mode positioned at a slice in the set of z-stacked images based on the second interaction.

### FIELD OF THE DISCLOSURE

This disclosure relates generally to improved image navigation techniques and, more particularly, to improved methods for momentum-based image navigation.

### BACKGROUND

Medical imaging enables the non-invasive visualization of the body's internal structures for purposes of diagnosis and disease treatment. The most common types of diagnostic and interventional radiology exams include computed tomography (CT) scans, fluoroscopy, and magnetic resonance imaging (MRI). Computed tomography (CT)-used to visualize organs, bones, soft tissue, and blood vessels-consists of an x-ray source rotating around a patient to produce cross-sectional images used for image reconstruction to generate the final 3D anatomical image. Fluoroscopy also utilizes an X-ray source, as well as a fluorescent screen, to enable real-time visualization of the patient for purposes of urological surgery, catheter placement, including vascular and cardiac treatments. Magnetic resonance imaging (MRI) uses a magnetic field in combination with radio waves, with multiple transmitted radiofrequency pulses applied in sequence to emphasize select tissues or abnormalities. Increases in the number of diagnostic medical procedures and high prevalence of chronic diseases continue to raise the global demand for medical imaging modalities, advanced diagnostic image processing and analysis software, as well as more technically-advanced healthcare information technology (IT) systems.

### BRIEF SUMMARY

Certain examples provide apparatus, systems, and methods to navigate through a set of z-stacked images.

Certain examples provide a visualization processor comprising a position tracker to track movement of a pointing device with respect to a set of z-stacked images, a momentum detector to identify momentum applied to the pointing device in a first interaction based on a speed of navigation through the set of z-stacked images and configure navigation through the set of z-stacked images in a momentum-based mode based on the speed of navigation through the set of z-stacked images, and a navigation mode detector to detect a second interaction with the pointing device and exit a navigation mode and enter a normal mode positioned at a slice in the set of z-stacked images based on the second interaction.

Certain examples provide a computer-implemented method to navigate through a set of z-stacked images, the method comprising tracking movement of a pointing device with respect to the set of z-stacked images, identifying momentum applied to the pointing device in a first interaction based on a speed of navigation through the set of z-stacked images, configuring navigation through the set of z-stacked images in a momentum-based mode based on the speed of navigation through the set of z-stacked images, detecting a second interaction with the pointing device, and exiting a navigation mode and entering a normal mode positioned at a slice in the set of z-stacked images based on the second interaction.

Certain examples provide at least one computer readable storage medium including instructions which, when executed, cause at least one processor to at least track movement of a pointing device with respect to a set of z-stacked images, identify momentum applied to the pointing device in a first interaction based on a speed of navigation through the set of z-stacked images, configure navigation through the set of z-stacked images in a momentum-based mode based on the speed of navigation through the set of z-stacked images, detect a second interaction with the pointing device, and exit a navigation mode and enter a normal mode positioned at a slice in the set of z-stacked images based on the second interaction.

### BRIEF DESCRIPTION OF THE DRAWINGS

FIG. 1 illustrates an example z-stack navigation system.

FIG. 2 illustrates an example implementation of the visualization processor of the system of FIG. 1.

FIG. 3 illustrates a flow diagram of an example method of using a pointing device for momentum-based z-stack image navigation in accordance with the systems and or/apparatus of FIGS. 1-2 and example navigation of FIGS. 6 and 9.

FIG. 4 illustrates a flow diagram of an example method of using a pointing device for momentum-based z-stack image navigation with a scroll-wheel in accordance with the systems and/or apparatus of FIGS. 1-2 and example navigation of FIG. 6.

FIG. 5 illustrates an example data flow diagram illustrating the navigation of z-stack images using scroll-wheel generated momentum based on the z-stack navigation system devices of FIGS. 1 and/or 2.

FIG. 6 illustrates an example navigation of z-stacks using momentum generated by a scroll-wheel in accordance with the systems and or/apparatus of FIGS. 1-2, flow diagrams of FIGS. 3-4, and example data flow of FIG. 5.

FIG. 7 illustrates a flow diagram of an example method of using a pointing device for momentum-based z-stack image navigation with a cursor in accordance with the systems and/or apparatus of FIGS. 1-2 and example navigation of FIG. 9.

FIG. 8 illustrates an example data flow diagram illustrating the navigation of z-stack images using cursor-generated momentum based on the z-stack navigation system devices of FIGS. 1 and/or 2.

FIG. 9 illustrates an example navigation of z-stacks using momentum generated by a cursor in accordance with the system of FIGS. 1-2, flowcharts of FIGS. 3 and 7, and example data flow of FIG. 8.

FIG. 10 illustrates a flow diagram of an example method of using a pointing device for dynamic scrolling of z-stack images in accordance with the systems and/or apparatus of FIGS. 1-2 and example navigation of FIG. 9.

FIG. 11 illustrates an example data flow diagram illustrating the navigation of z-stack images using dynamic scrolling based on the z-stack navigation system devices of FIGS. 1 and/or 2.

FIG. 12 illustrates an example navigation of z-stacks using dynamic scrolling by a cursor in accordance with the system of FIGS. 1-2, flowcharts of FIGS. 3 and 10, and example data flow of FIG. 11.

FIG. 13 is a block diagram of a processor platform structured to execute the example machine readable instructions of at least FIGS. 3-5, 7-8, and 10-11 to implement components disclosed and described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings. The figures are not to scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

While certain examples are described below in the context of medical or healthcare systems, other examples can be implemented outside the medical environment.

Medical imaging techniques used most commonly for purposes of diagnostic and interventional radiology include computed tomography (CT) and magnetic resonance imaging (MRI). Given the large amount of data involved in the processing of medical images and the increasing quality of this medical imaging data, the storage, exchange, and transmission of medical images most commonly relies on the use of the Digital Imaging and Communication in Medicine (DICOM) Standard, which incorporates international standards of image compression, visualization, presentation, and exchange. The storage and transmission of imaging data is possible due to the compression of DICOM imaging data files. For example, CT scanners can simultaneously acquire as many as 320 slices during each rotation of the X-ray tube, with a thin-slice CT scan dataset consisting of over 500 image slices, an abdominal CT scan alone generating up to 1,000 images. A CT exam of a thorax producing sub-millimeter image slice thickness with high in-plane resolution can yield up to 600 MB-1 GB of data.

Radiologists utilize stack mode viewing of cross-sectional images, also known as z-stacks, to navigate through the volumetric data sets, the 2D image slices presented in sequence along the z-axis. Navigating through the z-stacks as opposed to viewing the individual images in tile-mode introduces motion of the image slices that can be controlled by a user based on interactions with a computer mouse and/or other pointing device. For example, a user may use the mouse scroll-wheel or click and drag the cursor to scroll through the images more quickly. However, navigation through the z-stacks can be time consuming and tedious. There is a need for improved systems and methods that provide the user with a greater range of motion through the image slices and enhanced speed of navigation. Improved z-stack navigation has a number of advantages including: (1) improved speed of navigation allows a user to more quickly identify an area of interest or navigate to a particular region of interest, (2) the process of image navigation becomes less cumbersome and more intuitive for the user, and (3) improved navigation yields reductions in time and cost associated with assessment of medical imaging data.

Example systems and methods disclosed herein allow for a user to navigate through z-stacks using a pointing device that translates a momentum applied to the pointing device by the user into a corresponding speed of navigation through the z-stacks. Example systems and methods disclosed herein also allow a user to end the navigation prior to cycling through all the z-stacks that could be covered by the momentum applied through the pointing device. If the pointing device is a computer mouse, for example, systems and methods disclosed herein allow the user to apply momentum-based navigation to the z-stacks via a scroll-wheel or a “click & drag” of the cursor.

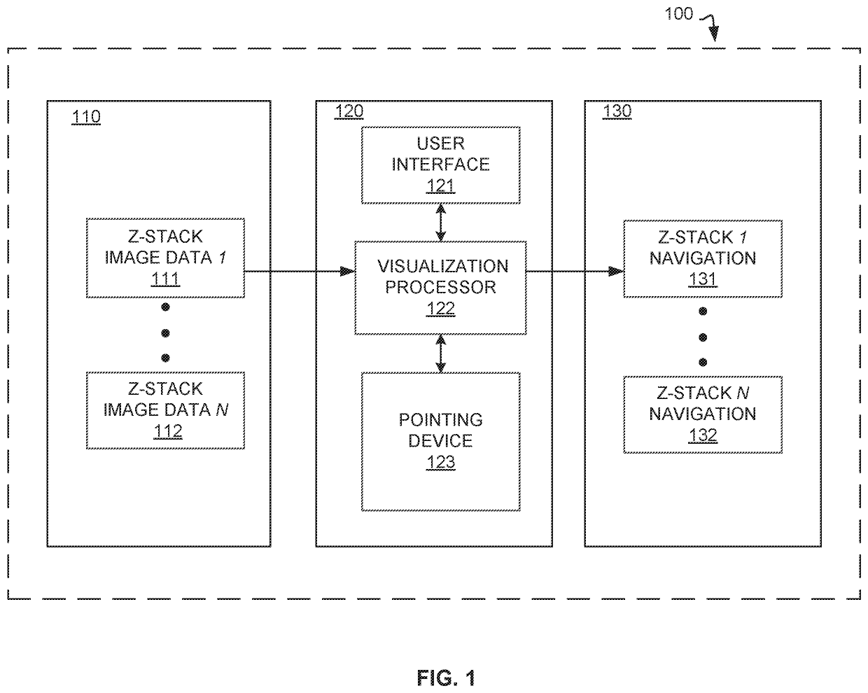

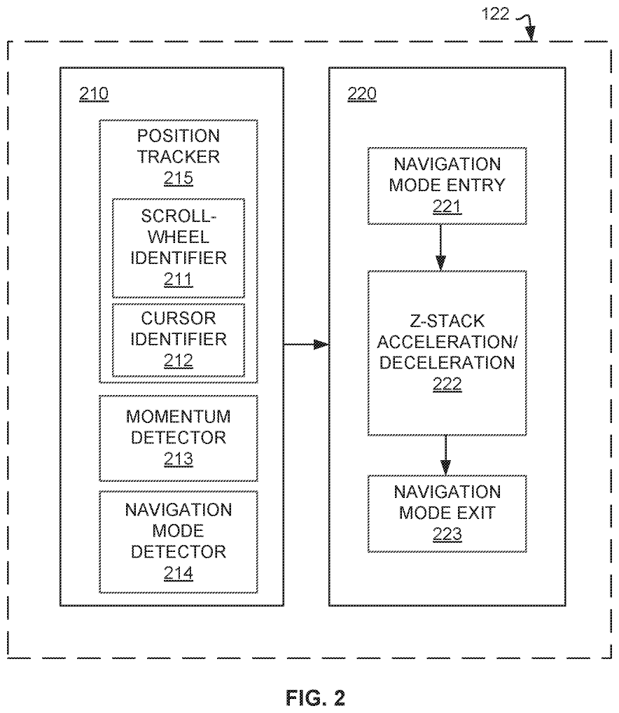

FIG. 1 illustrates an example z-stack navigation system 100 including one or more image data inputs 110. For example, the image data input 110 can include a z-stack from a three-dimensional (3D) input image data file 111 (e.g., a computed tomography (CT) image, a magnetic resonance (MR) image, an ultrasound image, an x-ray image, a positron emission tomography (PET) image, etc.). The one or more 3D images can show an inner portion or anatomy of a human body, for example. As shown in FIG. 1, one or more z-stack image data files 111, 112 can be provided as input. The example system 100 also includes a pointing device processor 120 to construct a user interface driver 121 that allows a user to interact with a pointing device interface 123 and a visualization processor 122. The pointing device interface 123 receives input from, and provides output to, the pointing device (e.g., a computer mouse, touchpad, touchscreen, other cursor-moving device, etc.). The combination of the user interface driver 121, pointing device interface 123, and visualization processor 122 elements allows for the rapid processing of z-stack image data file 111 to enable navigation through the z-stacks based on user interaction with the pointing device interface 123. The elements of the pointing device processor 120 are described in further detail with respect to FIG. 2 below.

The example system 100 also includes a user interface output generator 130 to provide an output from the pointing device processor 120. For example, the user interface output generator 130 provides the z-stack navigation output 131, and any additional z-stack navigation outputs 132 resulting from the input of z-stack image data 111, 112. The visualization processor 122 receives inputs from the pointing device interface 123 that cause the processor 122 to process the input and drive output on the user interface driver 121, resulting in the movement of the images generated as a result of z-stack navigation outputs 131, 132. In some examples, multiple z-stacks can be navigated through simultaneously if the pointing device is engaged to process user interaction with the device while multiple separate z-stack image data files 111, 112 are being visualized.

FIG. 2 illustrates an example implementation of the visualization processor 122 of the system 100. The visualization processor 122 receives input from the pointing device interface 123 that cause the process 122 to provide output to the pointing device interface 123, which interacts with the pointing device. A pointing device can be any device that can be initiated by the user to serve as an input interface to perform a motion that can be input as spatial data (e.g., continuous, multi-dimensional, etc.) into a computer. For example, the pointing device can include, but is not limited to, motion-tracking devices such as a computer mouse, trackpoint, trackball, joystick, pointing stick, or a finger-tracking device. The pointing device can also include, but is not limited to, a touch-sensitive surface (e.g., touchpad, graphics tablet, touchscreen, etc.) that uses input from a user based on touch generated innately by the user (e.g., using a finger, etc.) or through a separate device (e.g., a stylus, a pen, etc.) that can also be used to provide motion-based or touch sensitive input to any computing device used to navigate through z-stack images. Movement parameters generated by the pointing device interface 123 during a user's interaction with the pointing device are translated into a momentum-based motion used to navigate through consecutive images in the z-stack. The movement parameters can include the speed, duration, and directionality of the pointing device movement, for example. These parameters are translated by the processor into instructions to be executed by the processor to modify content displayed by the user interface driver 121.

Standard z-stack image navigation involves a cursor or other position indicator being manipulated by a computer mouse or scroll-wheel on a computer mouse or keypad to scroll or otherwise move through a stack of images such that display of the image slices iterates through the z-stack at a speed corresponding to (e.g., proportional to or otherwise consistent with) the “click & drag” cursor movement or the rotation of the scroll-wheel. However, the motion translated from computer mouse-based input to the z-stack image data set has to be generated in real-time, as opposed to being applied and allowed to take effect over a defined period of time. Therefore, navigation through a large data file can take a much longer period of scrolling and dragging by the user than necessary. In contrast, example methods and systems disclosed herein provide a technologically improved z-stack image navigation system 100 that enables a more time-efficient method of navigation by allowing the user to apply a motion to the pointing device that continues to traverse the z-stack image slices even once a user is no longer applying a motion to a pointing device in real-time.

As shown in FIG. 2, a data structure driving the visualization processor 122 includes an input configurator 210 and a navigation mode controller 220. Input to the navigation mode controller 220 is provided by the input configurator 210 which includes a position tracker 215 (including a scroll-wheel identifier 211 and a cursor identifier 212), a momentum detector 213, and a navigation mode detector 214. The configurator 210 utilizes the scroll-wheel identifier 211 to determine motion generated by the scroll-wheel, the cursor identifier 212 to determine cursor position with respect to the z-stack image data input 111, the momentum detector 213 to translate the movement parameters generated by the pointing device interface 123 into the momentum applied to the z-stacks, and the navigation mode detector 214 to determine whether the pointing device is in a navigation mode or a normal mode. The navigation mode controller 220 controls the pointing device interface 123 status to determine whether the pointing device is in navigation mode entry 221, z-stack acceleration/deceleration 222, or navigation mode exit 223. The navigation mode entry 221 corresponds to an enabling of the pointing device interface 123 to provide movement parameters to the momentum detector 213, which translates pointing device movements (e.g., from movement of the pointing device itself, from movement of a mouse scroll wheel or other secondary interface associated with the pointing device, etc.) into corresponding navigation through the z-stack of images.

For example, when a user engages the computer mouse in a “click & hold” position, this indicates the entry of the pointing device (e.g., computer mouse, trackpad, etc.) into a navigation mode (e.g., navigation mode entry 221). If a user then proceeds to move the cursor, the cursor identifier 212 detects the motion and the momentum detector 213 uses the movement parameters generated by the cursor motion to initiate z-stack acceleration/deceleration 222. Navigation through the z-stacks using this pointing device-generated (e.g., scroll-wheel, cursor) motion is a momentum scroll. Once the user ceases to “click & drag” the mouse, the pointing device interface 123 performs a navigation mode exit 223 and no momentum is detected using the momentum detector 213 even though the cursor can still be in motion following the end of the “click & drag” event. Another example of a navigation mode exit 223 can be initiated when a user initially employs the “click & drag” method to scroll through the z-stacks and then decides to stop the navigation by clicking the mouse. A click of the mouse and/or other pointing device (e.g., a selection of a mouse button, a depression of a scroll wheel, a depression of a touch pad interface, etc.) during navigation of the z-stacks results in navigation mode exit 223, ending the z-stack scroll at the z-stack image slice where the mouse click was applied by the user. The change in mode allows the user to navigate to a specific part of the z-stack of images by engaging the momentum scroll to navigate through the regions that are not of interest until a z-stack image slice of interest is identified. Similarly, if a scroll-wheel is engaged by the user, the event that initiates entry into the navigation mode can be, for example, the position of the cursor on the z-stack image data plane. Once the user begins to scroll, the scroll-wheel can move smoothly or advance between notches or stops defined with respect to positions on the scroll-wheel, and the rotation of the scroll-wheel is detected by the scroll-wheel identifier 211. The momentum detector 213 detects the momentum applied through the scroll-wheel (e.g., recording movement parameters) to translate the scroll-wheel based motion into a momentum-based navigation through the z-stacks. When the scroll-wheel is no longer engaged by the user, the pointing device interface 123 undergoes a navigation mode exit 223.

In practice, a change in momentum is determined based on an amount of force applied to an object and a length of time that the force is applied to the object, for example. The momentum detector 213 determines the velocity of the pointing device over the period of time that the pointing device is in navigation mode (e.g., the device movement used to navigate through the z-stack). Changes in pointing device velocity correspond to acceleration/deceleration during navigation through the z-stack image slices. Momentum-based navigation allows the continued traversal of image slices within the z-stack of images until all the images have been traversed, for example. Using momentum-based navigation, the images of the z-stack are traversed even after the pointing device has exited the navigation mode. The rate of change of the image slices therefore depends directly on the input motion from the pointing device interface 123, such that navigation through the z-stacks decreases at a rate consistent with total duration of the momentum that is applied through the pointing device. For example, a “click & drag” motion of the mouse in navigation mode performed with an initial acceleration results in an increased speed of navigation through the z-stack slices, followed by a decreasing speed of navigation through the image slices as momentum dissipates, eventually causing navigation through the z-stack of image slices to stop due to exhaustion of the applied momentum.

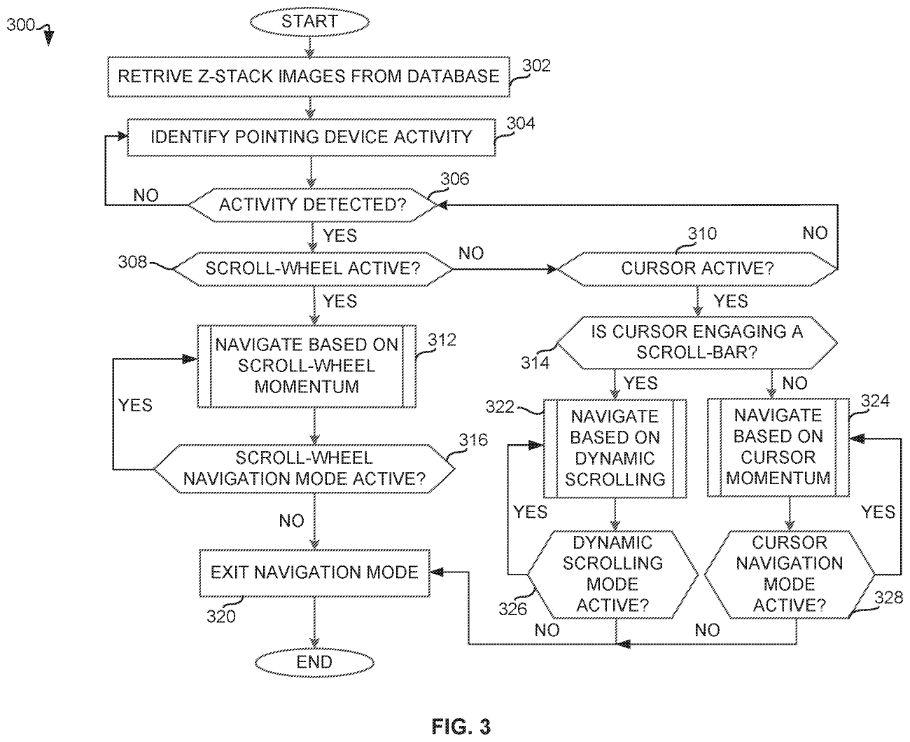

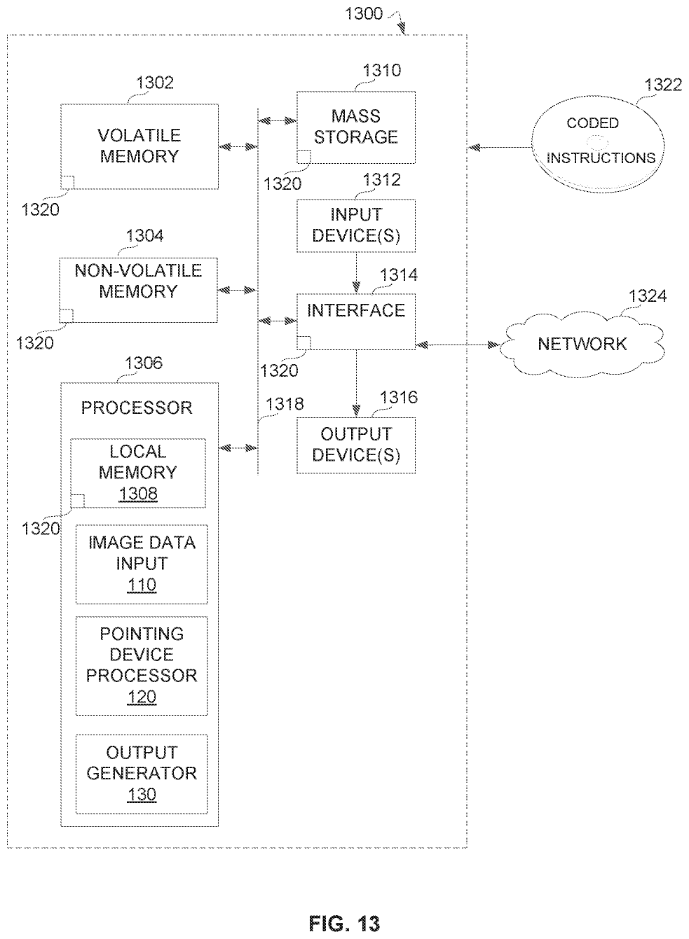

A flowchart representative of example machine readable instructions for implementing components disclosed and described herein are shown in conjunction with at least FIG. 13. In the examples, the machine readable instructions include a program for execution by a processor such as the processor 1306 shown in the example processor platform 1300 discussed below in connection with FIGS. 3-12. The program may be embodied in machine readable instructions stored on a tangible computer readable storage medium such as a CD-ROM, a floppy disk, a hard drive, a digital versatile disk (DVD), a Blu-ray disk, or a memory associated with the processor 1306, but the entire program and/or parts thereof could alternatively be executed by a device other than the processor 1306 and/or embodied in firmware or dedicated hardware. Further, although the example program is described with reference to the flowcharts and data flows illustrated in conjunction with at least FIGS. 3-5, 7-8, and 10-11 many other methods of implementing the components disclosed and described herein may alternatively be used. For example, the order of execution of the blocks may be changed, and/or some of the blocks described may be changed, eliminated, or combined. Although the flowcharts and data flows of at least FIGS. 3-5, 7-8, and 10-11 depict example operations in an illustrated order, these operations are not exhaustive and are not limited to the illustrated order. In addition, various changes and modifications may be made by one skilled in the art within the spirit and scope of the disclosure. For example, blocks illustrated in the flowcharts may be performed in an alternative order or may be performed in parallel.

As mentioned above, the example process(es) of at least FIGS. 3-5, 7-8, and 10-11 may be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example process(es) of at least FIGS. 3-5, 7-8, and 10-11 can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. In addition, the term “including” is open-ended in the same manner as the term “comprising” is open-ended.

As shown in the example method 300 depicted in FIG. 3, momentum-based z-stack image navigation can be performed using a pointing device. At block 302, z-stack images are retrieved from a database. For example, the visualization processor 122 retrieves the image data input 110 from a database. At block 304, pointing device activity is identified. For example, the data structure driving pointing device interface 123 activity determines whether the scroll-wheel identifier 211 or the cursor identifier 212 detects any scroll-wheel or cursor-based activity, respectively. If activity is detected at block 306, a determination of whether the scroll-wheel is active is made at block 308. If no activity is detected at block 306, the data structure driving the pointing device interface 123 continues to scout for activity from the scroll-wheel identifier 211 or the cursor identifier 212. If the scroll-wheel identifier 211 is not active at block 308, the cursor identifier 212 is checked for activity at block 310. In the presence of scroll-wheel activity at block 308, the z-stack navigation proceeds based on scroll-wheel momentum at block 312. In the presence of cursor activity at block 310, the z-stack navigation proceeds based on cursor momentum at block 314. For example, momentum detector 213 is used to translate the scroll wheel-based or cursor-based motion into momentum-based navigation through the z-stack image. Once the scroll-wheel navigation mode (block 316) or the cursor navigation mode (block 318) is determined to be inactive, the navigation mode is exited at block 320. For example, navigation mode entry 221 is performed when there is a detection of scroll-wheel or cursor-based activity by the pointing device interface 123 at blocks 308 or 310. Navigation through the z-stack takes place in response to momentum detector 213 input into the pointing device processor 120, corresponding to z-stack acceleration/deceleration 222. Once the navigation mode detector 214 determines there is no longer any activity to proceed with scroll-wheel navigation (block 316) or cursor-based navigation (318), navigation mode exit 223 is initiated at block 320. Thus, either movement of a scroll-wheel or movement of a pointing device itself can drive momentum-based navigation through a z-stack of images.

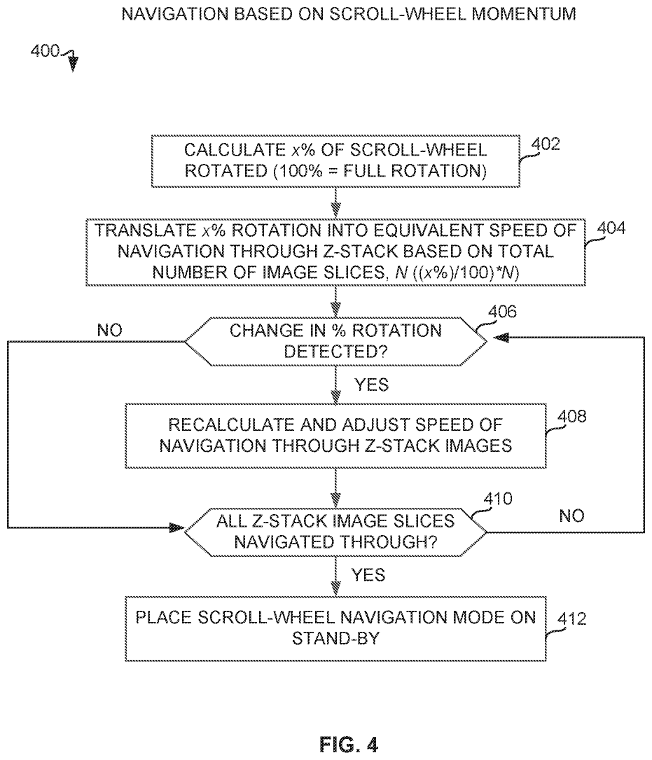

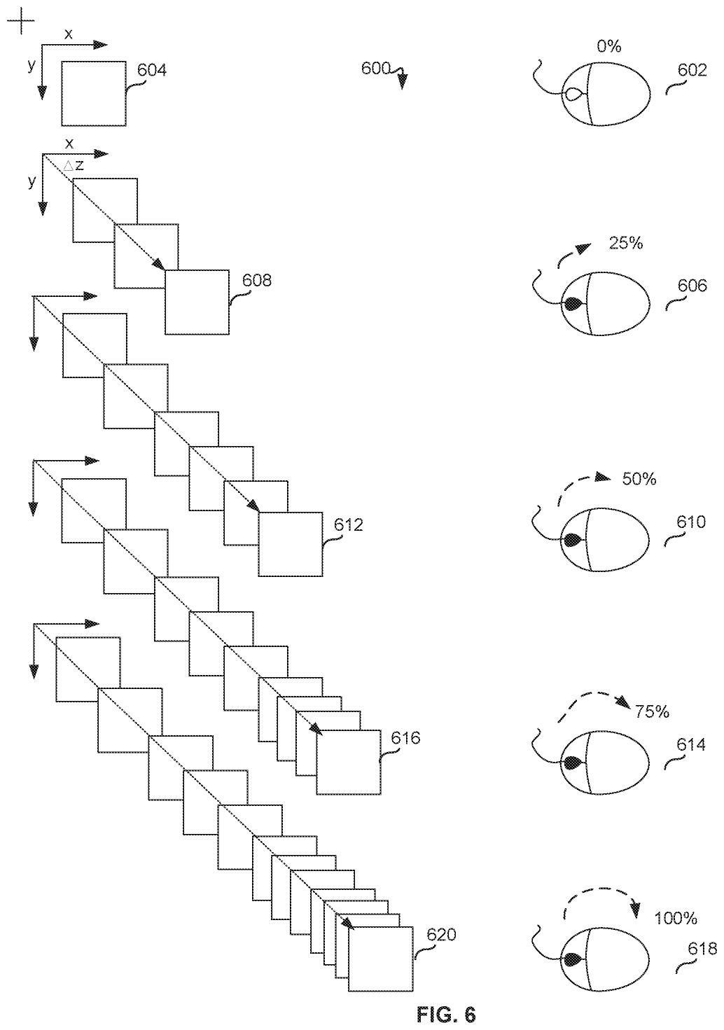

FIG. 4 is a flowchart representative of an example method 400 of using a pointing device for momentum-based z-stack image navigation with a scroll-wheel in accordance with the systems and or/apparatus of FIGS. 1-2 and example navigation of FIG. 6. Navigation using the scroll-wheel is hereafter referred to as a scrolling event. At block 402, the percentage of the scroll-wheel rotated (x %) during a scrolling event is calculated based on the scroll-wheel identifier 211 input to the pointing device processor 120, which uses the information in combination with the momentum detector 213 input to proceed with z-stack navigation. At block 404, the percentage of rotation (x %) is translated into the equivalent speed of navigation through the z-stack based on the total number of image slices in the z-stack (e.g., N ((x %)/100)*N=total number of slices to scroll through, where N=total number of image slices). In one example, a full rotation of the scroll-wheel during the scrolling event can indicate that all of the z-stack image slices are to be navigated through, unless the user initiates a backwards rotation of the scroll-wheel to backtrack in the z-stack image slice sequence. In some examples, the total number of z-stack slices that are navigated during a full-rotation of the scroll-wheel can be adjusted based on the total z-stack file size (e.g., a larger data set would require two full rotations of the scroll-wheel to initiate the navigation of the full set of z-stack image slices). At block 406, the change in % rotation of the scroll-wheel is detected and initiates a recalculation of the total z-stack images that need to be navigated through (block 408). The speed of the navigation during the scrolling event is also adjusted based on the amount of time it takes for the user to make the change in scroll-wheel rotation. At block 410, the system 100 determines whether all z-stack image slices have been navigated through based on the calculated number of slices (N) at block 408. If the navigation is complete, scroll-wheel navigation mode is placed on stand-by (block 412) using the navigation mode controller 220.

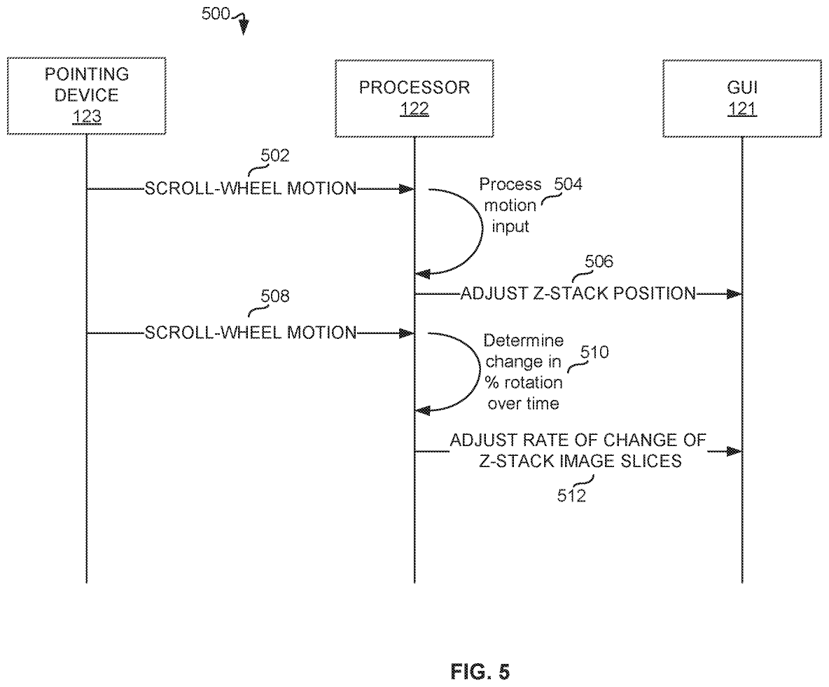

In the illustrated example of FIG. 5 using data flow 500, when example scroll-wheel motion 502 is initiated by the example pointing device interface 123, the example input motion is processed 504 by the example visualization processor 122. The navigation of the z-stack is initiated through the adjustment of the example z-stack position 506, shown to the user via the example graphical user interface (GUI) driver 121. For example, an initial entry into the navigation mode by the scroll-wheel can involve the user clicking the scroll-wheel before rotating it. Once the scroll-wheel motion causes the entry of the example pointing device interface 123 into navigation mode through an initial interaction with the example user interface driver 121, further example scroll-wheel motions 508 are used to determine the change in the % of rotation over time 510 during the scrolling event. This engages the example momentum detector 213 to determine the momentum to apply during navigation through the z-stacks, adjusting the rate of change of the z-stack image slices 512.

FIG. 6 illustrates an example navigation 600 of z-stacks using momentum generated by a scroll-wheel in accordance with the systems of FIGS. 1-2, flowcharts of FIGS. 3-4 and example data flow of FIG. 5. If an example scroll-wheel has not been engaged by the user (0% rotation), the example user interface shows only one image slice within the z-stack 604. Once the user has engaged the scroll-wheel resulting in a rotation of 25% (606), the navigation through the z-stack is initiated such that the z-stack slices 608 are navigated through based on their image slice sequence. Likewise, if the percentage of the scroll-wheel being rotated increases to 50% (610) or 75% (614), the number of image slices navigated through increases (612 and 616, respectively), with the increased percentage of rotation also corresponding to an increase in the speed of navigation, the image slices in 616 appearing more quickly within areas that correspond to the equivalent increase in the percentage of scroll-wheel rotation. For example, the later z-stack image slices in 616 appear more rapidly than those in the beginning of the stack, seen in 608 and 612. In some examples, the scroll-wheel rotation can correspond to the ‘notches’ the wheel is rotated, each ‘notch’ of rotation corresponding to a defined percentage of scroll-wheel rotation. The complete 180 degree rotation of the scroll-wheel (618) initiates the navigation of the z-stack slices at a maximum speed. For example, the image slices in 620 appear rapidly towards the end of the stack due to the scroll-wheel rotation being changed from a 75% rotation to a 100% rotation.

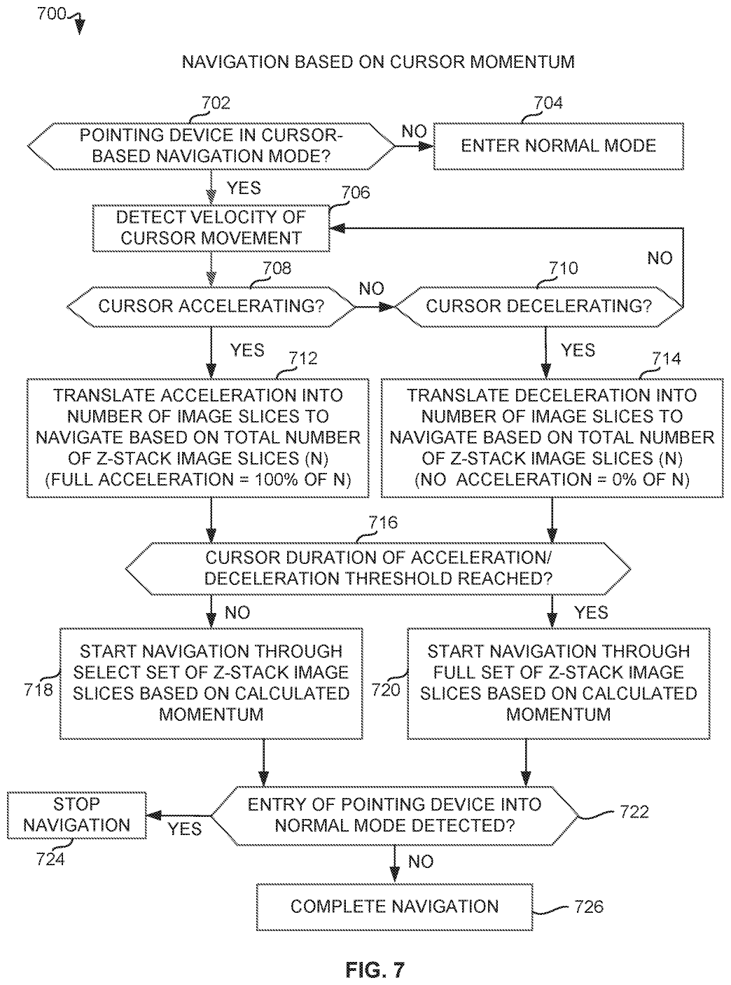

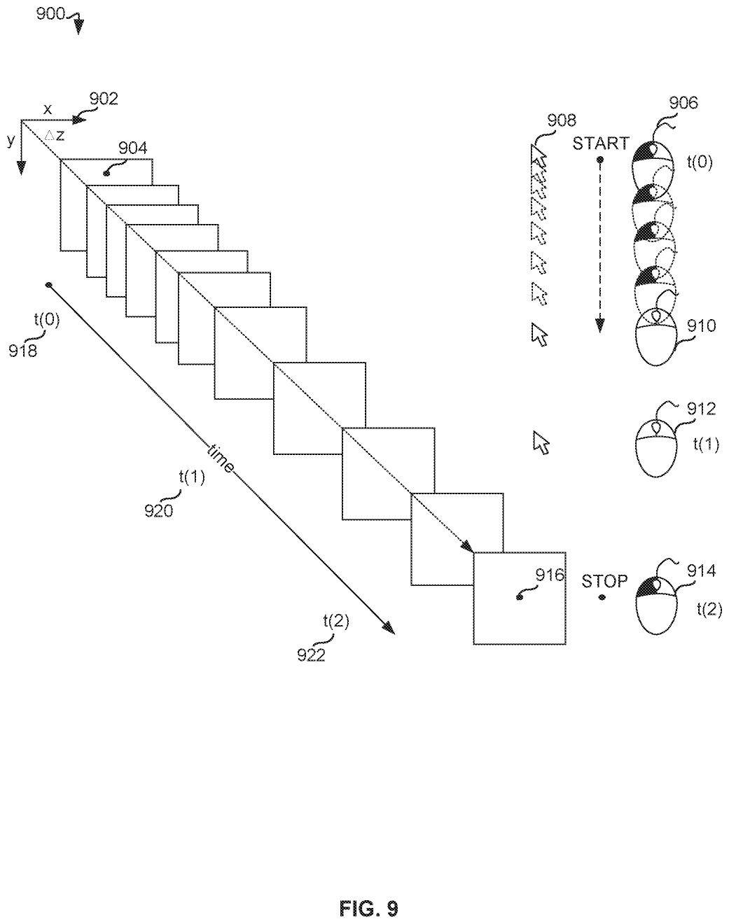

FIG. 7 is a flowchart representative of an example method 700 of using a pointing device for momentum-based z-stack image navigation using a cursor in accordance with the systems and or/apparatus of FIGS. 1-2 and example navigation of FIG. 9. At block 702, the z-stack navigation system checks whether the pointing device is in cursor-based navigation mode using the cursor identifier 212. In some examples, such as when the pointing device is a computer mouse, the mouse is engaged in a “click & drag” motion, hereafter referred to as a dragging event, to enable cursor-based navigation. For example, if the dragging event is not detected, the cursor-based navigation mode enters a normal mode (block 704). In the presence of cursor movement, the velocity of cursor motion is detected at block 706. In some examples, if the cursor is being dragged upwards or downwards, the directionality combined with the applied speed of cursor dragging corresponds to the forward or backwards navigation through the z-stack. The identification of cursor acceleration 708 or deceleration 710 using the momentum detector 213 corresponds to a respective rate of navigation through the z-stacks based on the total number of z-stack image slices (N). For example, at full acceleration of the cursor over a defined period of time, 100% of the image slices within the stack are navigated through. At block 712/714, the acceleration/deceleration of the cursor is translated into the total number of image slices that are to be traversed. At block 716, the momentum identifier 213 is used to determine whether the cursor reached an acceleration/deceleration threshold. For example, if the duration of the cursor dragging movement exceeds a predefined number of seconds, the entire z-stack will be navigated through (block 720). If the duration of cursor movement does not meet or exceed the set threshold, a select number of image slices are shown (block 718), determined based on the duration of the momentum applied through the pointing device. At block 722, the navigation mode detector 214 is checked to determine whether the pointing device has entered a normal mode (e.g., left the navigation mode). In some examples, if the user has performed a single click using the mouse button during the course of the navigation through the z-stack, resulting in a mouse button engagement, the pointing device enters a normal mode, such that navigation is ceased at the z-stack image slice during which the mouse click occurred (block 724). In some examples, if the user makes no further movements with the pointing device after the movement(s) that prompts the initial navigation through the z-stack, the images continue to be shown sequentially, the presentation of the images decelerating as the momentum-based navigation cycle comes to a completion (block 726).

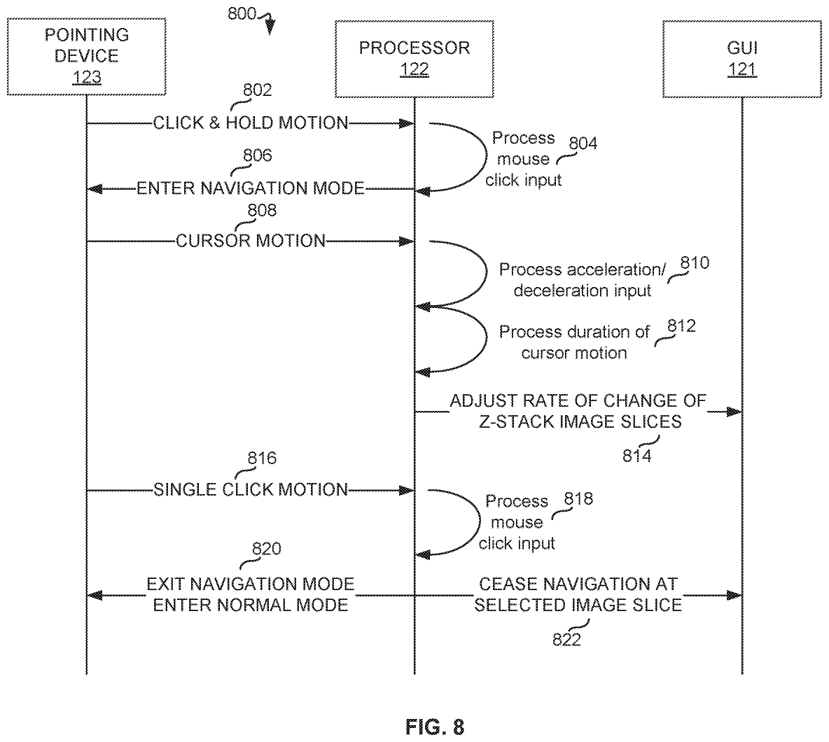

In the illustrated example of FIG. 8 using data flow 800, when the pointing device interface 123 generates a “click & hold” motion 802 (e.g., using a pointer, such as a computer mouse, etc.), a visualization processor 122 processes the mouse click input 804, causing the pointing device interface 123 to enter into navigation mode 806. After entry into the navigation mode, additional cursor motion 808 is tracked using the cursor identifier 212. The momentum detector 213 is used to process the acceleration/deceleration input 810 generated by the pointing device interface 123 and process the duration of the cursor motion 812. This information is translated into a momentum-based navigation through the z-stack, thereby adjusting the rate of change of the z-stack image slices 814 during navigation, the image slices displayed using the graphical user interface driver 121. Additional inputs from the mouse can initiate a change in the navigation mode using the navigation mode detector 214. For example, a single click motion 816 causes the visualization processor 122 to process the mouse click input 818, cause a navigation mode exit and a normal mode entry 820. This action causes navigation through the z-stack using the dragging event to stop at the z-stack image slice 822 that was shown using the user interface driver 121 when the user engaged the mouse in the single-click motion 816. In some examples, the navigation mode can change to a normal mode through an abrupt transition (e.g., a click of the mouse or release of the pointing device, etc.). In some examples, the navigation mode can change to a normal mode through a gradual transition (e.g., momentum slows after mouse cursor movement of the dragging event stops, etc.).

FIG. 9 illustrates an example navigation 900 of z-stacks using momentum generated by a cursor in accordance with the system of FIGS. 1-2, flowcharts of FIG. 3 and FIG. 7, and example data flow of FIG. 8. An example computer mouse enters navigation mode once the user has left-clicked the mouse button 906, resulting in a mouse button engagement, to allow for dragging event-based scrolling through the z-stack image. The cursor 908 is tracked using the cursor identifier 212. The initial mouse-clicking motion 906 results in the “click & hold” motion of the dragging event that causes the selection of the z-stack image slice 904 where the navigation begins. The dragging of the cursor generates a velocity that also provides input of the directionality of motion (e.g., dragging the mouse downwards at a certain speed). The duration of the dragging motion also provides input to the momentum detector 213 that generates the transfer of the momentum applied to the pointing device from the pointing device to the z-stack. In an example disengagement of the dragging event, the user stops pressing on the mouse button 910. The momentum detector 213 can then process the velocity and duration of the dragging event motion to determine the total duration 922 of the momentum-based scrolling through the z-stack based on the initial start of the motion at 918. If no further interaction with the pointing device occurs 912, navigation through the z-stack gradually slows to a stop 922 through deceleration. For example, if the mouse is not being engaged 912 after the applied motion 906-910, scrolling through the sequential z-stack image slices continues at 920. In some examples, an abrupt exit from the navigation mode can occur if the user engages the pointing device (e.g., left-click of the mouse button), which causes the navigation to cease at the z-stack image slice 916 where the motion was applied. In some examples, the momentum-based navigation of multiple z-stacks may include temporal navigation through the z-stacks. In some example, the temporal navigation through the z-stacks permits the user to compare medical imaging data generated at different time points.

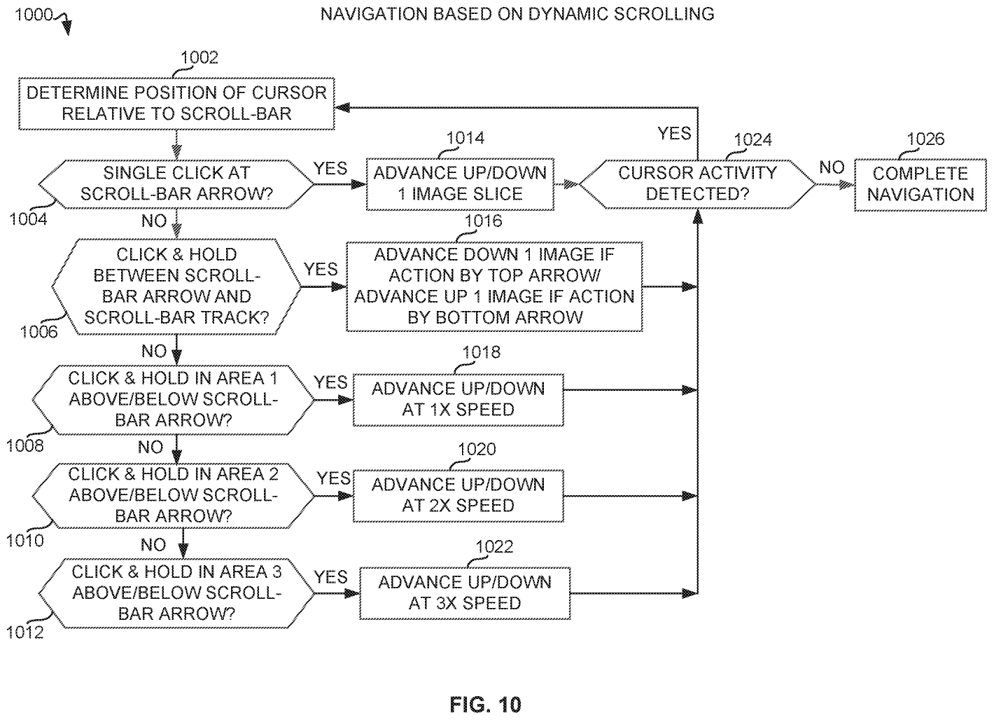

FIG. 10 illustrates a flow diagram 1000 of an example method of using a pointing device for dynamic scrolling of z-stack images in accordance with the systems and/or apparatus of FIGS. 1-2 and example navigation of FIG. 9. Dynamic scrolling allows for the user to navigate z-stack images by interacting with a scroll-bar (e.g., with a graphical user interface scroll bar element and/or other interactive control, etc.). For example, the user can engage a computer mouse by clicking and holding down on the mouse button, hereafter referred to as a holding event, and position the cursor in the vicinity of the scroll-bar to navigate through the z-stack. The scroll-bar user interface 121 permits the user to navigate at different speeds through the z-stack of images based on the areas surrounding the scroll-bar with which the pointing device, such as a cursor, interacts. For example, at block 1002, the position of a cursor relative to a scroll-bar is determined to optimize the response of the navigation based on, for example, whether the cursor is in closer proximity to the top arrow or the bottom arrow of the scroll bar. For example, if the cursor is positioned closer to the top arrow of a scroll-bar, the user is more likely to scroll upwards rather than downwards. At block 1004, if the cursor is used to perform a single-click at the scroll-bar arrow, then, at block 1014, this action causes the advancement of the z-stack by one image slice either up or down, depending on whether the up or down scroll-bar arrow is clicked using the pointing device 123. If, at block 1006, the motion performed by the pointing device 123 is instead a “click & hold” motion that occurs between the scroll-bar arrow and the scroll-bar track, then, at block 1016, the image advances up or down by one z-stack image slice, depending on whether the action is performed in the vicinity of the bottom arrow or the top arrow of the scroll-bar, respectively. If, at block 1008, the motion performed is a holding event in the first area above or below the scroll-bar up/down arrow, then, at block 1018, the z-stack image slices are navigated at a 1× speed (e.g., a “normal” speed) either upwards through the z-stack (if the area the cursor is positioned at during the “hold” action is above the top arrow) or downwards through the z-stack (if the area the cursor is positioned at during the “hold” action is below the bottom scroll-bar arrow).

If, at block 1010, the motion performed by the cursor of the pointing device 123 is a holding event in the second area above or below the scroll-bar up/down arrow, then, at block 1020, the z-stack image slices are navigated at a 2× speed (e.g., double speed) upwards/downwards through the z-stack. If, at block 1012, the motion performed is a holding event in the third area above or below the scroll-bar up/down arrow, then, at block 1022, the z-stack image slices are navigated at a 3× speed (e.g., triple speed) upwards/downwards through the z-stack. Therefore, the holding event location can determine the speed of navigation through the z-stack image slices. Once the holding event-based cursor motion of the pointing device 123 is no longer detected at block 1024, navigation through the z-stack is complete at block 1026. If the user continues to interact with the scroll-bar using the pointing device 123, then the position of the cursor relative to the scroll-bar is again determined and processed at block 1002 until cursor activity is no longer detected at block 1024. In some examples, during navigation of z-stack images using dynamic scrolling as described here, the momentum detector 213 can be engaged to determine how long the cursor, for example, of the pointing device 123 is performing the holding event in a designated area of the scroll-bar in order to calculate how many images to navigate through, similar to the dragging event motion of navigation based on cursor momentum.

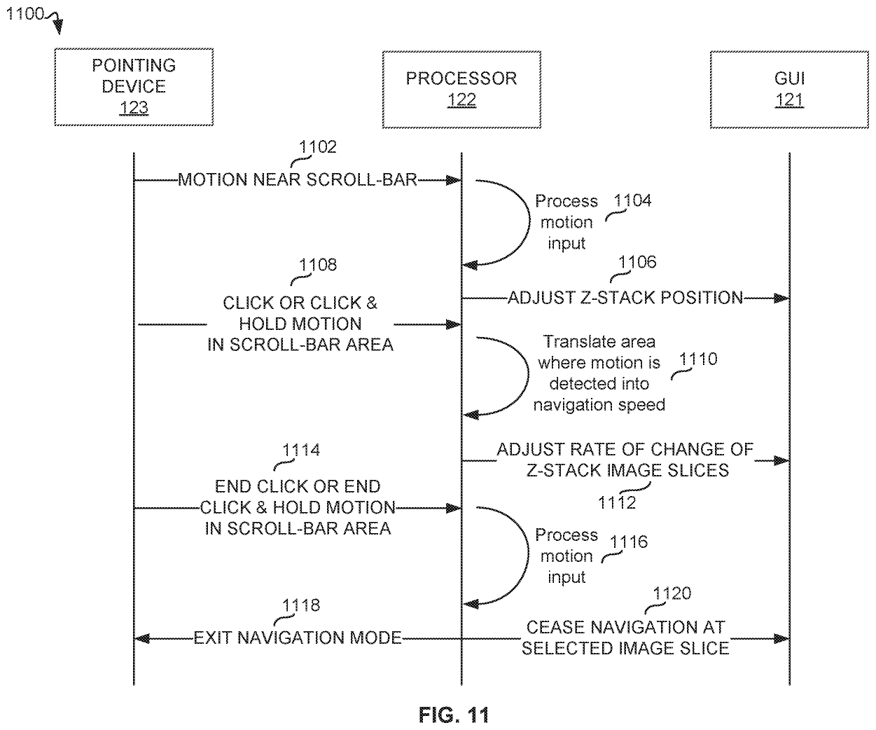

FIG. 11 illustrates an example data flow diagram 1100 illustrating the navigation of z-stack images using dynamic scrolling based on the z-stack navigation system devices of FIGS. 1 and/or 2. During user interaction with the pointing device 123 component, such as a cursor, motion near the scroll-bar 1102 is detected and processed 1104 by the processor 122. In some examples, based on this initial interaction, the z-stack position can be adjusted 1106 so as to bring the z-stack images to the first or last image slice. Once the pointing device 123 performs a single click or a holding event in the scroll-bar area, the area where the motion occurs and its proximity to the top or bottom scroll-bar arrow determines the speed of navigation 1110 through the z-stack image slices using the processor 122, as described in FIG. 10. As a result, the rate of change through the z-stack image slices is adjusted at 1112, as shown to the user with the GUI 121. Once the pointing device 123 is no longer engaged (e.g., the holding event in the scroll-bar area is no longer detected 1114) this change in user interaction with the pointing device 123 is processed 1116 and the navigation mode is exited 1118 and the GUI 121 can display the last image slice that is shown after the navigation is complete 1120.

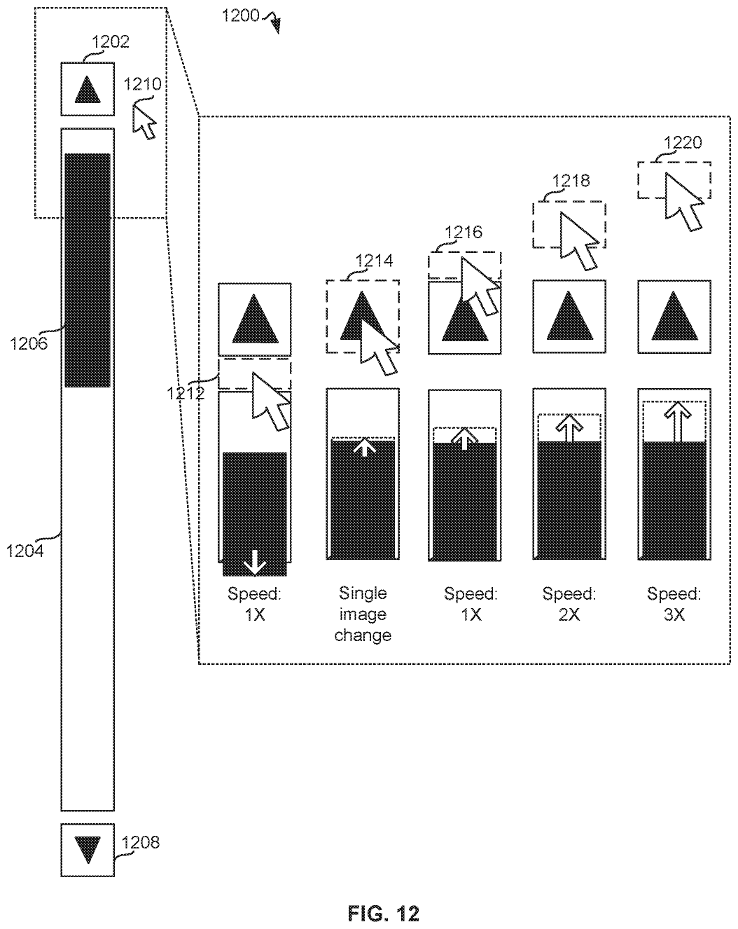

FIG. 12 illustrates an example navigation 1200 of z-stacks using dynamic scrolling by a cursor in accordance with the system of FIGS. 1-2, flowcharts of FIGS. 3 and 10, and example data flow of FIG. 11. An example scroll-bar includes a top arrow 1202, a bottom arrow 1208, a track 1204, and a slider 1206. The slider 1206 can move along the scroll-bar track 1204 depending on the pointing device 123 interaction with the example cursor 1210. For example, the cursor 1210 can be positioned between the scroll-bar top arrow 1202 or the scroll-bar down arrow 1208 and the scroll-bar track 1204, in the area 1212. If the pointing device 123 engages the cursor 1210 based on user interaction such that the cursor 1210 performs a holding event in the 1212 area, the slider 1206 moves at a 1× speed through the z-stack image slices downwards if the cursor 1210 is positioned below the top arrow 1202 or upwards if the cursor 1210 is positioned above the bottom arrow 1208. If the pointing device 123 is instead positioned at the top arrow 1202 or the bottom arrow 1208 and pointing device 123 performs a single clicking motion 1214, the slider 1206 moves such that the z-stack image slices advance by a single image either upwards through the z-stack (e.g., if the cursor 1210 is clicking on the top arrow 1202) or downwards through the z-stack (e.g., if the cursor 1210 is clicking on the bottom arrow 1208). If the cursor 1210 is instead positioned at the first area 1216 adjacent to the top arrow 1202 of the scroll-bar, the z-stack images are navigated through at a speed of 1× upwards through the stacks. If the cursor 1210 is positioned at the second area 1218 adjacent to the top arrow 1202 of the scroll-bar, the z-stack images are navigated through at a speed of 2x upwards through the stacks. If the cursor 1210 is positioned at the third area 1220 adjacent to the top arrow 1202 of the scroll-bar, the z-stack images are navigated through at a speed of 3x upwards through the stacks. In some examples, these same first, second, and third areas can be activated in the areas below the bottom arrow 1208 of the scroll-bar, with the same speeds of navigation applied downwards through the z-stacks. In some examples, when the cursor 1202 ceases the holding event in the designated scroll-bar areas, the z-stack image navigation halts at a specific image. In some examples, the momentum detector 213 can be used to apply momentum-based navigation to the dynamic scrolling navigation action to allow the z-stack images to continue being navigated through sequentially even when the holding event is no longer active, depending on the length of time that the holding event was applied by the cursor 1210 of the pointing device 123.

While example implementations are illustrated in conjunction with FIGS. 1-12, elements, processes and/or devices illustrated in conjunction with FIGS. 1-12 can be combined, divided, re-arranged, omitted, eliminated and/or implemented in any other way. Further, components disclosed and described herein can be implemented by hardware, machine readable instructions, software, firmware and/or any combination of hardware, machine readable instructions, software and/or firmware. Thus, for example, components disclosed and described herein can be implemented by analog and/or digital circuit(s), logic circuit(s), programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the components is/are hereby expressly defined to include a tangible computer readable storage device or storage disk such as a memory, a digital versatile disk (DVD), a compact disk (CD), a Blu-ray disk, etc. storing the software and/or firmware.

FIG. 13 is a block diagram of an example processor platform 1300 structured to executing the instructions of at least FIGS. 3-5, 7-8, and 10-11 to implement the example components disclosed and described herein. The processor platform 1300 can be, for example, a server, a personal computer, a mobile device (e.g., a cell phone, a smart phone, a tablet such as an iPad™), a personal digital assistant (PDA), an Internet appliance, or any other type of computing device.

The processor platform 1300 of the illustrated example includes a processor 1306. The processor 1306 of the illustrated example is hardware. For example, the processor 1306 can be implemented by integrated circuits, logic circuits, microprocessors or controllers from any desired family or manufacturer.

The processor 1306 of the illustrated example includes a local memory 1308 (e.g., a cache). The example processor 1306 of FIG. 3000 executes the instructions of at least FIGS. 3-5, 7-8, and 10-11 to implement the systems, infrastructure, displays, and associated methods of FIGS. 1-12 such as the example image data inputs 110, pointing device processor 120 (and its user interface driver 121, pointing device interface 123, visualization processor 122), user interface output generator 130, etc. The processor 1306 of the illustrated example is in communication with a main memory including a volatile memory 1302 and a non-volatile memory 1304 via a bus 1318. The volatile memory 1302 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 1304 may be implemented by flash memory and/or any other desired type of memory device. Access to the main memory 1302, 1304 is controlled by a clock controller.

The processor platform 1300 of the illustrated example also includes an interface circuit 1314. The interface circuit 1314 may be implemented by any type of interface standard, such as an Ethernet interface, a universal serial bus (USB), and/or a PCI express interface.

In the illustrated example, one or more input devices 1316 are connected to the interface circuit 1314. The input device(s) 1312 permit(s) a user to enter data and commands into the processor 1306. The input device(s) can be implemented by, for example, a sensor, a microphone, a camera (still or video, RGB or depth, etc.), a keyboard, a button, a mouse, a touchscreen, a track-pad, a trackball, isopoint and/or a voice recognition system.

One or more output devices 1316 are also connected to the interface circuit 1314 of the illustrated example. The output devices 1312 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 1314 of the illustrated example, thus, typically includes a graphics driver card, a graphics driver chip or a graphics driver processor.

The interface circuit 1314 of the illustrated example also includes a communication device such as a transmitter, a receiver, a transceiver, a modem and/or network interface card to facilitate exchange of data with external machines (e.g., computing devices of any kind) via a network 1324 (e.g., an Ethernet connection, a digital subscriber line (DSL), a telephone line, coaxial cable, a cellular telephone system, etc.).

The processor platform 1306 of the illustrated example also includes one or more mass storage devices 1310 for storing software and/or data. Examples of such mass storage devices 1310 include floppy disk drives, hard drive disks, compact disk drives, Blu-ray disk drives, RAID systems, and digital versatile disk (DVD) drives.

The coded instructions 1322 of FIG. 10 may be stored in the mass storage device 1310, in the volatile memory 1302, in the non-volatile memory 1304, and/or on a removable tangible computer readable storage medium such as a CD or DVD.

From the foregoing, it will be appreciated that the above disclosed apparatus, systems, and methods have been disclosed to generate a pointing device interface and visualization processor for purposes of navigating through a set of z-stacked images. Certain examples improve navigation through z-stacks in a time-efficient manner by processing momentum applied by a user to a pointing device and transferring it to the z-stack images through identification of movement parameters, including the velocity and duration of motion. Certain examples improve the navigation of z-stacks by allowing the user to apply a motion to the pointing device that continues to traverse the z-stack image slices even once a user is no longer applying a motion to a pointing device in real-time. Certain examples drive improvements in the control of z-stack navigation by allowing the user to navigate to a specific part of the z-stack of images by engaging the momentum scroll to navigate through the regions that are not of interest until a z-stack image slice of interest is identified.

Certain examples enable a user to navigate multiple z-stacks through simultaneously if the pointing device is engaged to process user interaction with the device while multiple separate z-stack image data files are being visualized. Certain examples permit the user to engage momentum scroll through the use of the scroll-wheel, or the use of the cursor in a “click & drag” motion. In certain examples, a “click & drag” motion of the mouse in navigation mode performed with an initial acceleration results in an increased speed of navigation through the z-stack slices, followed by a decreasing speed of navigation through the image slices as momentum dissipates. In certain examples, if the user makes no further movements with the pointing device after the movement(s) that prompts the initial navigation through the z-stack, the images continue to be shown sequentially, the presentation of the images decelerating as the momentum-based navigation cycle comes to a completion. In certain examples, the user can navigate a z-stack using dynamic scrolling, which allows the detection of the area where a cursor motion is occurring in the vicinity of a scroll-bar to determine the rate of change of z-stack image slices.

Although certain example methods, apparatus and systems have been described herein, the scope of coverage of this patent is not limited thereto. On the contrary, this patent covers all methods, apparatus and systems fairly falling within the scope of the claims of this patent.

1. A visualization processor, comprising:
a position tracker to track movement of a pointing device with respect to a set of z-stacked images;
a momentum detector to:
identify momentum applied to the pointing device in a first interaction based on a speed of navigation through the set of z-stacked images, wherein the speed of navigation is determined based at least in part on a number of image slices in the set of z-stacked images and an amount of movement detected by the pointing device; and
configure navigation through the set of z-stacked images in a momentum-based mode based on the speed of navigation through the set of z-stacked images; and
a navigation mode detector to:
detect a second interaction with the pointing device; and
exit a navigation mode and enter a normal mode positioned at one of the image slices in the set of z-stacked images based on the second interaction.

2. The visualization processor of claim 1, wherein the momentum detector is to identify momentum applied to the pointing device in a first interaction including momentum detected through at least one of a dragging event, a scrolling event, or a holding event applied through at least one of a computer mouse-based input or a touch sensitive input.

3. The visualization processor of claim 2, wherein momentum detected through the scrolling event includes scrolling resulting from turning of a mouse scroll wheel, a percentage of rotation applied to the scroll wheel during the scrolling event used to determine a number of z-stacked image slices to navigate through.

4. The visualization processor of claim 2, wherein momentum detected through the dragging event adjusts based on a duration of acceleration or deceleration applied through the pointing device, the set of z-stacked image traversed completely once a duration of acceleration or deceleration threshold reached.

5. The visualization processor of claim 2, wherein momentum detected through the holding event includes holding resulting from a mouse button engagement, a duration of the holding event and a location of the holding event relative to a scroll-bar used to determine the speed of navigation through the z-stacked image slices.

6. The visualization processor of claim 1, wherein the momentum detector is to navigate through the set of z-stacked images in momentum-based mode, traverse consecutive images, and display the consecutive images as a pointer associated with the pointing device moves through the set of z-stacked images.

7. The visualization processor of claim 1, wherein the speed of navigation through the set of z-stacked images is determined based on a rate of acceleration and deceleration applied on a plane through the pointing device.

8. The visualization processor of claim 1, wherein the speed of navigation through the set of z-stacked images during which momentum is no longer applied to the pointing device decreases at a rate consistent with total duration of the applied momentum.

9. The visualization processor of claim 1, wherein the second interaction comprises at least one of a second selection using a computer mouse or a release of the pointing device.

10. A computer-implemented method to navigate through a set of z-stacked images, the method comprising:
tracking movement of a pointing device with respect to the set of z-stacked images;
identifying momentum applied to the pointing device in a first interaction based on a speed of navigation through the set of z-stacked images, wherein the speed of navigation is determined based at least in part on a number of image slices in the set of z-stacked images and an amount of movement detected by the pointing device;
configuring navigation through the set of z-stacked images in a momentum-based mode based on the speed of navigation through the set of z-stacked images;
detecting a second interaction with the pointing device; and
exiting a navigation mode and entering a normal mode positioned at one of the image slices in the set of z-stacked images based on the second interaction.

11. The method of claim 10, wherein identifying momentum applied to the pointing device in a first interaction includes momentum detected through at least one of a dragging event, a scrolling event, or a holding event applied through at least one of a computer mouse-based input or a touch sensitive input.

12. The method of claim 11, wherein momentum detected through the scrolling event includes scrolling resulting from turning of a mouse scroll wheel, a percentage of rotation applied to the scroll wheel during the scrolling event used to determine a number of z-stacked image slices to navigate through.

13. The method of claim 11, wherein momentum detected through the dragging event adjusts based on a duration of acceleration or deceleration applied through the pointing device, the set of z-stacked images traversed once a duration of acceleration or deceleration threshold reached.

14. The method of claim 11, wherein momentum detected through the holding event includes holding resulting from a mouse button engagement, a duration of the holding event and a location of the holding event relative to a scroll-bar used to determine the speed of navigation through the z-stacked image slices.

15. The method of claim 10, wherein navigation through the set of z-stacked images in the momentum-based mode traverses consecutive images and displays the consecutive images as a pointer associated with the pointing device moves through the set of z-stacked images.

16. The method of claim 10, wherein the speed of navigation through the set of z-stacked images is determined based on a rate of acceleration and deceleration applied on a plane through the pointing device.

17. The method of claim 10, wherein the speed of navigation through the set of z-stacked images during which momentum is no longer applied to the pointing device decreases at a rate consistent with total duration of the applied momentum.

18. The method of claim 10, wherein the second interaction includes at least one of a second selection using a computer mouse or a release of the pointing device.

19. At least one computer readable storage medium including instructions which, when executed, cause at least one processor to at least:
track movement of a pointing device with respect to a set of z-stacked images;
identify momentum applied to the pointing device in a first interaction based on a speed of navigation through the set of z-stacked images, wherein the speed of navigation is determined based at least in part on a number of image slices in the set of z-stacked images and an amount of movement detected by the pointing device;
configure navigation through the set of z-stacked images in a momentum-based mode based on the speed of navigation through the set of z-stacked images;
detect a second interaction with the pointing device; and
exit a navigation mode and enter a normal mode positioned at one of the image slices in the set of z-stacked images based on the second interaction.

20. The computer readable storage medium of claim 19, wherein the instructions further cause the processor to identify momentum applied to the pointing device in a first interaction including momentum detected through at least one of a dragging event, a scrolling event, or a holding event applied through a computer mouse-based input or a touch sensitive input.

21. The computer readable storage medium of claim 20, wherein momentum detected through the scrolling event includes scrolling resulting from turning of a mouse scroll wheel, a percentage of rotation applied to the scroll wheel during the scrolling event used to determine a number of z-stacked image slices to navigate through.

22. The computer readable storage medium of claim 20, wherein momentum detected through the dragging event adjusts based on a duration of acceleration or deceleration applied through the pointing device, the set of z-stacked image traversed completely once a duration of acceleration or deceleration threshold reached.

23. The computer readable storage medium of claim 20, wherein momentum detected through the holding event includes holding resulting from a mouse button engagement, a duration of the holding event and a location of the holding event relative to a scroll-bar used to determine the speed of navigation through the z-stacked image slices.

24. The computer readable storage medium of claim 19, wherein the instructions further cause the processor to navigate through the set of z-stacked images in the momentum-based mode, traverse consecutive images, and display the consecutive images as a pointer associated with the pointing device moves through the set of z-stacked images.

25. The computer readable storage medium of claim 19, wherein the speed of navigation through the set of z-stacked images is determined based on a rate of acceleration and deceleration applied on a plane through the pointing device.

26. The computer readable storage medium of claim 19, wherein the speed of navigation through the set of z-stacked images during which momentum is no longer applied to the pointing device decreases at a rate consistent with total duration of the applied momentum.

27. The computer readable storage medium of claim 19, wherein the set of z-stacked images comprises one or more two-dimensional cross sectional image slices from three-dimensional image data corresponding to a patient, and wherein the one or more two-dimensional cross sectional image slices are produced by an imaging device.
